(19) United States
(12) Patent Application Publication
(10) Pub. No.: US 2007/0184478 A1
(43) Pub. Date: Aug. 9, 2007

(54) Process for detecting a plurality of target nucleic acids

(76) Inventors: Walter Gumbrecht, Herzogenaurach (DE); Jorn Mosner, Erlangen (DE); Manfred Stanzel, Erlangen (DE); Christian Zilch, Leipzig (DE)

Correspondence Address: HARNESS, DICKEY & PIERCE, P.L.C., P.O.BOX 8910, RESTON, VA, 20195, US

(21) Appl. No.: 11/702,209

(22) Filed: Feb. 5, 2007

(30) Foreign Application Priority Data
Feb. 6, 2006 (DE) 10 2006 005 287.0

(51) Int. Cl. C12Q 1/68; C12M 1/34

(52) U.S. Cl. 435/6; 435/287.2

(57) ABSTRACT

A process and a kit for detecting a plurality of target nucleic acids are disclosed. In at least one embodiment, the process and/or kit includes using primers coupled to a semi-solid phase support.

### PRIORITY STATEMENT

The present application hereby claims priority under 35 U.S.C. §119 on German patent application number DE 10 2006 005 287.0 filed Feb. 6, 2006, the entire contents of which is hereby incorporated herein by reference.

1. Field

Embodiments of the invention generally relate to a process for detecting a plurality of target nucleic acids, by using primers coupled to a semi-solid phase support for example.

2. Background

Nucleic acid amplification processes have been disclosed in the prior art. The most commonly applied process is the polymerase chain reaction (PCR). This process enables nucleic acid molecules to be duplicated and is based on the replication of nucleic acids with the aid of thermostable polymerases. The process involves contacting a pair of oligonucleotide primers (single-stranded oligonucleotides) with the nucleic acid to be amplified. The primers are chosen so as to bind at the two ends on the complementary strands of a fragment to be amplified.

During elongation, one of the primers is then elongated in one direction and the other primer is elongated in the opposite direction, along the target nucleic acid in the 3′ direction (forward and reverse primers). Alternatively, forward and reverse primers are also referred to as sense and antisense primers. In this way, it is possible for the section located between the sites on the target nucleic acid, which are complementary to the primers, to be amplified. Advantageously, primers, nucleotides and other interfering components of the PCR mixture are removed from the PCR products for subsequent detection reactions.

The course of the PCR includes a plurality of thermocycles of in each case three steps: first, the double-stranded DNA present in the sample is heated in order to separate the strands (denaturation). The temperature is then lowered to enable the primers to anneal to the DNA single strands (annealing). In the last step, the polymerase fills the DNA section between the primers with the in each case complementary nucleotides (elongation). This cycle is typically run approx. 15-50 times.

Biochips measure the concentration or presence of biomolecules (e.g. DNA, proteins) in biological samples. In DNA microarrays, specific capture molecules are coupled at distinct sites (spots) on the surface of suitable supports (such as, for example, glass, plexiglass, silicon). The capture molecules are usually single-stranded oligonucleotides (15-40 base pairs) or, alternatively, single-stranded PCR products directed to specific target molecules in a sample to be examined. The single-stranded capture molecules hybridize to corresponding complementary single-stranded target molecules (for example of a PCR product) at a defined stringency (temperature, buffer conditions) and can be identified with the aid of various detection processes. Most often optical, electrical or magnetic detection processes are employed for this purpose.

All detection processes have in common the precondition that only single-stranded nucleic acids are capable of being bound by the capture molecules present on the microarray (except the exotic 3-stranded hybrids). This precondition is already met when RNA samples are being used. If, for example, specific DNA sequences are to be detected in genotyping and in the detection of mutations (SNP analysis), but also if cDNA is to be detected in expression profile experiments, then the previously double-stranded DNA must be split into its individual strands (denatured). A typical PCR product, cDNA or double-stranded DNA fragment generated by restriction enzymes is usually split into its individual strands by heating to approx. 95° C. As an alternative to or in support of thermal cleavage, it is possible to use highly alkaline agents such as sodium hydroxide solution, for example, to separate the strands.

A problem here has proved to be the possible reannealing of the separated single strands, particularly at low stringency. Especially during hybridization on a microarray, the process of reannealing competes with hybridization to the in each case specific capture molecules on the surface of said microarray. This process is disadvantageous to the sensitivity of the assay because reannealing can significantly reduce the hybridization of desired nucleic acid sequences. Frequently, in particular at low concentration, the sensitivity is insufficient for detecting the target sequences.

Another problem in microarray experiments with an upstream PCR is the fact that amplification is limited to a small number of product species. This is a particular problem specifically if there is a significant difference in the melting temperatures of the different PCR primer pairs or in the length of the resulting PCR products.

In these cases, some sequences are preferably amplified in a “multiplex PCR”, resulting in an imbalance in PCR product concentrations after a few PCR cycles. However, the uneven product ratios resulting from the PCR are found to be particularly disadvantageous for subsequent detection of the target molecules (PCR products) on the surface of a microarray.

The efficiency of a hybridization to specific capture molecules can be significantly increased by accumulating the target DNA single strands over the in each case complementary sequences. In order to accomplish this accumulation of single-stranded DNA during a PCR reaction, use is made in particular of two processes:
a) asymmetrical PCR
b) specific removal of a DNA single strand

Re a): in asymmetrical PCR, a primer (for example sense primer) is added to the reaction buffer at a substantially higher concentration than the corresponding primer (for example antisense primer), with both of said primers being required for generating a PCR product. In the course of a PCR reaction, i.e. after a certain number of temperature cycles, both double-stranded and single-stranded DNA products are present, the latter, however, at a substantially higher concentration. In this case, separating the strands by temperature-induced or alkaline denaturation is not absolutely necessary. A process which uses a first primer pair and a further secondary primer at different concentrations is disclosed, for example, in the patent DE 198 02 905 C2.

Re b): the specific removal of a single strand usually involves providing a primer with a marker, for example a biotin molecule. In the subsequent PCR reaction one strand of the double-stranded DNA molecule is terminally biotinylated. Biotin is known to have a very high affinity for streptavidin, binding tightly to the latter. Streptavidin here is located on a phase such as magnetic beads, for example. After the DNA double strands have been denatured to single strands, the biotinylated single strand can subsequently be removed from the unlabeled single strand with the aid of the streptavidin-coupled magnetic beads binding the former and by applying a magnetic field. Alternatively, the biotinylated single strands can be removed by passing over streptavidin bound to a solid phase or a resin. This process has previously been described in the patent EP 0 418 960 A2 by Eastman Kodak.

After the above-described processes, the reaction products may be used further directly for hybridization to capture molecules on a microarray. Both processes have the advantage over a symmetrical PCR that the fixed capture molecules cannot compete with single-stranded DNA strands complementary to the target sequence which are present freely in the hybridization solution. Hybridization of the target sequences to the specific capture molecules is therefore much more efficient than after a symmetrical PCR reaction and, as a result, requires a shorter hybridization time. The overall assay time may thus be accelerated considerably by shortening the hybridization.

However, the processes described also have some substantial disadvantages:

The fundamental problem in asymmetrical PCR is the fact that determining the concentration of forward and reverse primers in order to achieve the optimal yield of single-stranded DNA is very complicated. The parameters of a PCR reaction which is determined, apart from the concentration, by magnesium ions, free nucleotides, template concentration and the free primers are additionally influenced by the number and length of each individual temperature cycle and by the temperature. These many different influential parameters must be optimized in time-consuming experiments in order to achieve the yields required for a microarray experiment.

In addition, divergent parameters apply in each case to different target sequences and the primer pairs used. The complexity of such an assay for detecting a plurality of different target sequences to be generated by a single PCR reaction (multiplex PCR) in particular increases significantly. The multiplex capability is usually exhausted when the number of different PCR products reaches about a dozen. This limitation considerably restricts the usage of a microarray for detecting multiple biological parameters, despite a large number of applied capture molecules.

The problem described proves to be less serious in the second method, namely biotinylation of a primer for PCR and subsequent removal via binding to streptavidin. However, there are also disadvantages here. However, firstly the amount of multiplexing is likewise very limited, secondly additional process steps are required in order to separate the two single strands of a DNA double strand permanently. This makes additional requirements to the microfluidics of an integrated system.

All processes have the problem of the PCR products not being pure but contaminated with primers, nucleotides, enzymes etc. and therefore usually requiring purification.

### SUMMARY

In at least one embodiment of the present invention, a detection process is provided for nucleic acids which overcomes at least partially the above-described disadvantages.

In at least one embodiment, a process for detecting a plurality of target nucleic acids coprises:
(i) providing a plurality of primer pairs consisting of in each case a first and a second oligonucleotide primer suitable for amplifying said target nucleic acids, with a plurality of said first primers being coupled to a semi-solid phase support and the in each case second primer being free,
(ii) providing a solution comprising a target nucleic acid to be detected,
(iii) contacting the primer pairs of (i) with the solution of (ii) and conducting an amplification reaction under conditions in which the target nucleic acid is amplified,
(iv) denaturing the double-stranded amplification products of (iii) to give single strands,
(v) removing the semi-solid phase support with single-stranded amplification products coupled thereto and/or with non-extended primers coupled thereto, and
(vi) detecting the target nucleic acids.

Optionally, step (iii) may be followed by a washing step to remove the soluble components of the PCR reaction mix (nucleotides, primers, enzymes, auxiliary substances etc.). The first and/or the second primer may advantageously be labeled.

According to an example embodiment, the first primer is present not only coupled to the semi-solid phase support but also free in solution, for example at low concentration. The presence of the first primer in a free, unbound form is beneficial to an efficient start of amplification.

In at least one embodiment of the present invention further relates to a kit for detecting a plurality of target nucleic acids, comprising
(a) a plurality of primer pairs consisting of in each case a first and a second oligonucleotide primer suitable for amplifying said target nucleic acid, with a plurality of said first primers being coupled to a semi-solid phase support and the in each case second primer being free, and
(b) optionally reagents for carrying out an amplification reaction.

The target nucleic acid may be a double-stranded nucleic acid, for example DNA, cDNA, etc., or else single-stranded nucleic acids such as RNA, with the complementary strand being completed prior to the reaction (e.g. by reverse transcription), where appropriate.

This process of at least one embodiment of the invention is suitable for complex assay systems, in particular for PCR, in particular multiplex PCR. At least one embodiment of the invention proposes the usage of a semi-solid phase support on which in each case specific primers are located for such an assay that requires a PCR reaction or multiplex PCR reaction. A suitable semi-solid phase support is in principle any granular material, with preference being given in particular to beads. The latter are particularly preferably magnetic beads. Such materials have already been disclosed in the prior art, for example epoxy-modified magnetic beads (Dynal).

### BRIEF DESCRIPTION OF THE DRAWINGS

The invention will be described in more detail on the basis of example embodiments with reference to the accompanying drawings in which:

FIG. 1 depicts a diagrammatic representation of target sequence 2 and first primers 4 which are provided with a label 10, and second primers 6 which are coupled to a magnetic bead 8.

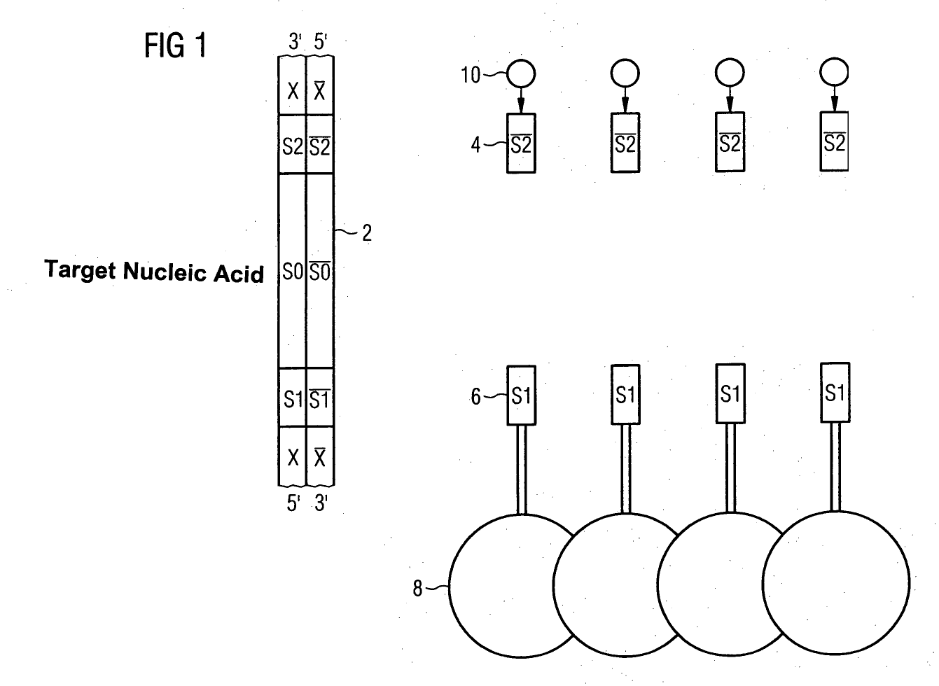

FIG. 2 diagrammatically depicts the double-stranded bead-bound amplification products after amplification.

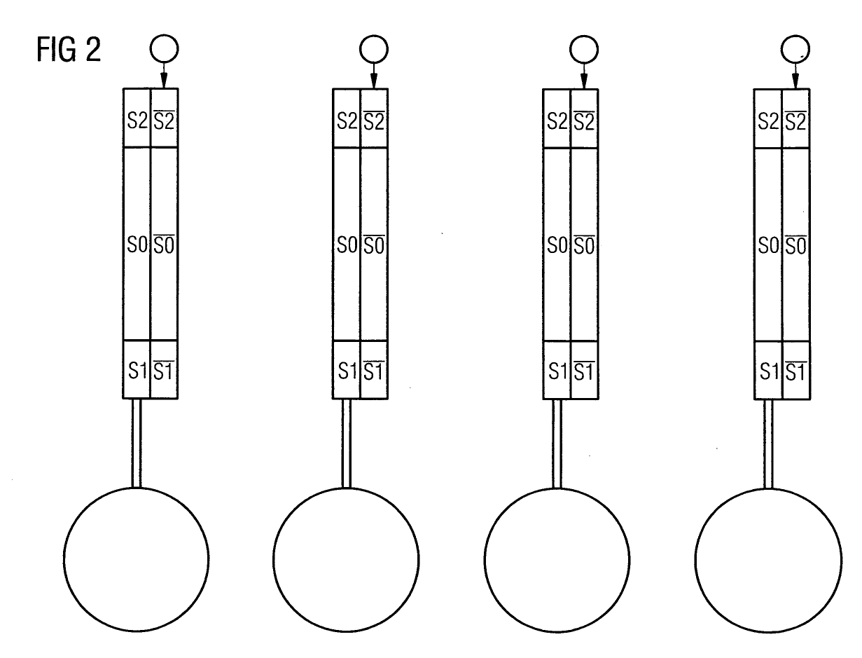

FIG. 3 diagrammatically depicts the single-stranded amplification products after denaturation of the double-stranded amplification products.

FIG. 4 diagrammatically depicts magnetic separation of the bead-bound single-stranded amplification products from the labeled double-stranded amplification products.

FIG. 5 diagrammatically depicts hybridization of the labeled single-stranded amplification products with complementary capture molecules 22 and detection thereof, which may be carried out, for example, optically or electrically or in any other manner.

FIG. 6 diagrammatically depicts an alternative hybridization of the bead-bound single-stranded amplification products with immobilized complementary capture molecules 22 and magnetic detection thereof.

FIG. 7 diagrammatically depicts a multiplex PCR reaction with different labeled free and bead-bound primers directed to various target sequences.

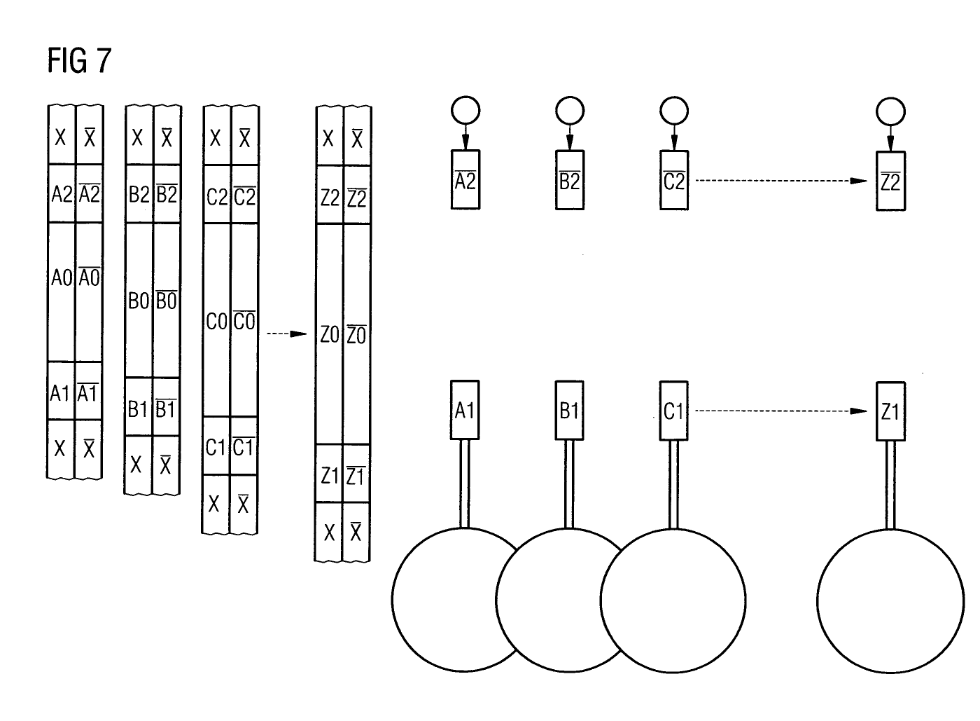

### DETAILED DESCRIPTION OF THE EXAMPLE EMBODIMENTS

It will be understood that if an element or layer is referred to as being “on”, “against”, “connected to”, or “coupled to” another element or layer, then it can be directly on, against, connected or coupled to the other element or layer, or intervening elements or layers may be present. In contrast, if an element is referred to as being “directly on”, “directly connected to”, or “directly coupled to” another element or layer, then there are no intervening elements or layers present. Like numbers refer to like elements throughout. As used herein, the term “and/or” includes any and all combinations of one or more of the associated listed items.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The terminology used herein is for the purpose of describing particular embodiments only and is not intended to be limiting of the present invention. As used herein, the singular forms “a”, “an”, and “the” are intended to include the plural forms as well, unless the context clearly indicates otherwise. It will be further understood that the terms “includes” and/or “including”, when used in this specification, specify the presence of stated features, integers, steps, operations, elements, and/or components, but do not preclude the presence or addition of one or more other features, integers, steps, operations, elements, components, and/or groups thereof.

In describing example embodiments illustrated in the drawings, specific terminology is employed for the sake of clarity. However, the disclosure of this patent specification is not intended to be limited to the specific terminology so selected and it is to be understood that each specific element includes all technical equivalents that operate in a similar manner.

Referencing the drawings, wherein like reference numerals designate identical or corresponding parts throughout the several views, example embodiments of the present patent application are hereafter described.

Each support particle, for example each individual magnetic bead, is provided with at least one specific type of oligonucleotide primer. In contrast, the corresponding second primers for the specific PCR products are free in solution. Part of the PCR reaction thus takes place on a kind of semi-solid phase (said magnetic bead). Since the beads can be moved during the reaction in order to facilitate diffusion, said PCR can by definition be referred to as semi-solid phase PCR. It is possible to choose between two variants of a PCR multiplex reaction using the semi-solid phase technology:

First variant: in each case one primer of the primer pair amplifying a specific sequence is located on one type of a magnetic bead (sense or antisense). The corresponding primer (antisense or sense) is free in the reaction solution. Thus there is only in each case one specific primer on a bead. A multiplex PCR uses X differently functionalized magnetic beads for X PCR products. The X corresponding primers are free in the reaction solution.

Second variant: more than one or all primers of a primer pair required for a multiplex PCR (sense or antisense) are located on one type of a magnetic bead. The in each case corresponding primers for the particular PCR product are free in the reaction solution. Therefore, all the beads used carry more than one function (multivalence) on the surface. A PCR multiplex with X different PCR products makes use of X primer pairs. All of a total of X different primers for one side (sense or antisense) of a DNA sequence to be amplified are on one type of a magnetic bead. The in each case corresponding X primers of each primer pair are again free in solution.

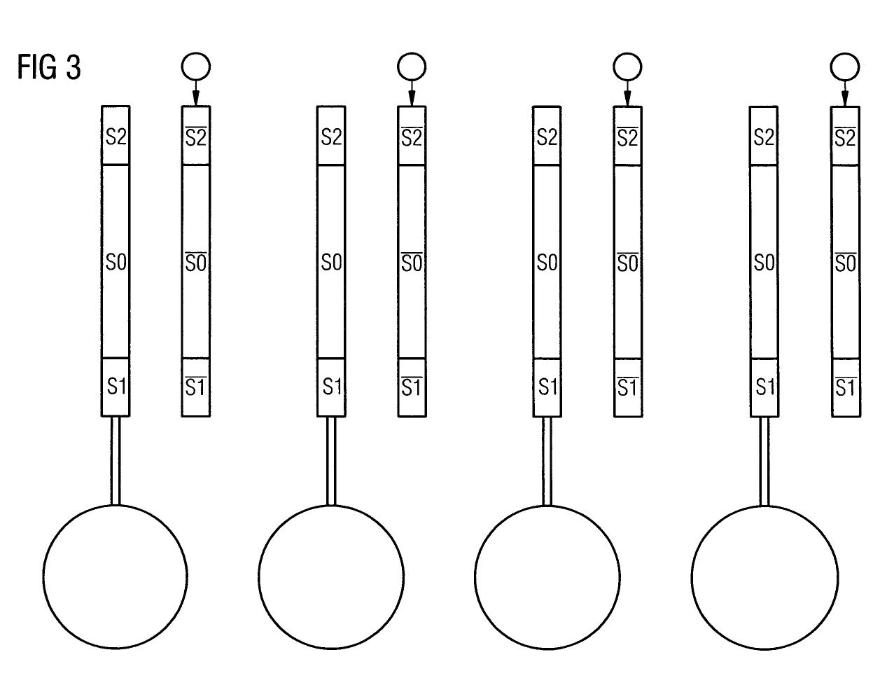

As the PCR reaction proceeds, in each case one DNA strand is elongated on the bead, with the other strand of a PCR product being elongated on the free primer. When the PCR reaction is completed, the majority of primers on the bead are in the double-stranded elongated form. If the double strands are then denatured, then one single strand of each PCR product will be free in solution, with the in each case complementary single strand being bound to the bead. This is depicted diagrammatically in FIG. 3, for example.

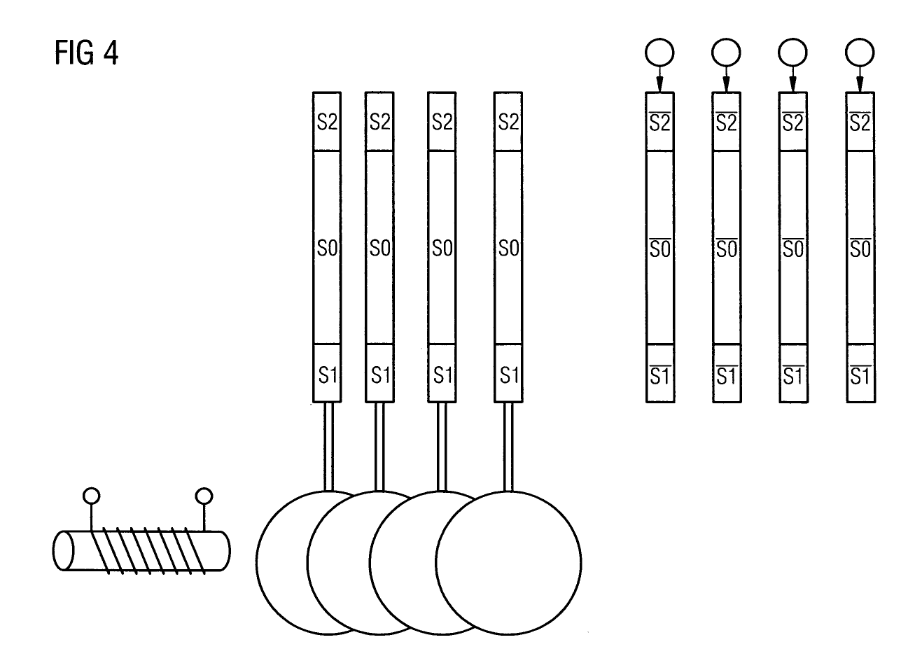

In a subsequent hybridization reaction on a microarray, it is then possible to retain all magnetic beads by means of a magnet in the PCR chamber and to transport the single-stranded PCR products free in solution to the hybridization chamber. The single-stranded PCR products which are still in solution then hybridize only with the complementary capture molecules located on the microarray because the complementary single strands on the bead cannot be reached any more. In this way, it becomes possible to implement a process equivalent to asymmetrical PCR, without having to carry out a complex titration of the corresponding primers. The removal is depicted diagrammatically in FIG. 4.

In addition, the primers may also be labeled. Suitable to this end are nucleic acid markers known in the prior art, such as, for example, biotin, fluorescent markers and the like. A particular advantage is for the second primer, i.e. the primer which is not coupled to the support, to be labeled. However, it is also possible for both primers of a primer pair to be labeled.

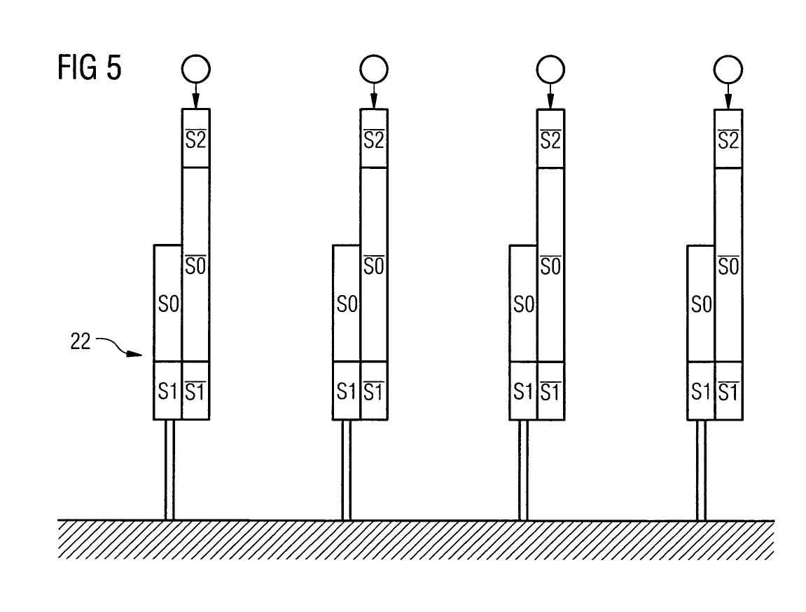

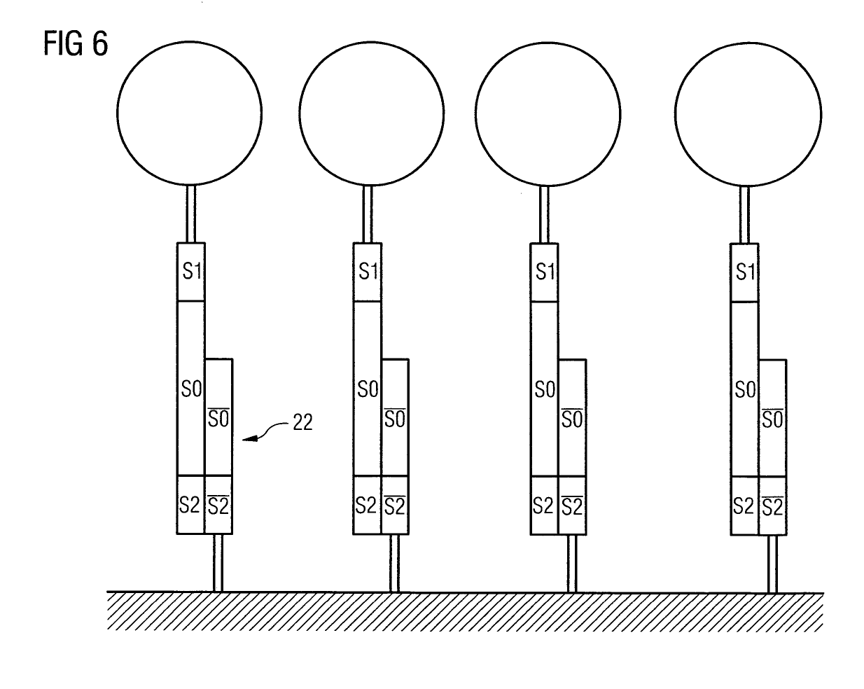

Following the removal, the amplified target nucleic acid is detected. This may be accomplished in several ways. One possibility is detection by way of hybridization with specific capture molecules which, for example, may be labeled or immobilized on a solid support. It is possible here to detect the strands amplified with either the first primer or the second primer. In one variant, said single strands are labeled, and in a second variant they are the primer-elongated single strands coupled to the beads. These variants are depicted, for example, in FIGS. 5 and 6.

A useful modification of an embodiment of the invention includes amplification (PCR), hybridization on the microarray and detection of the hybridization events in a chamber. In the case of a single chamber, after completion of the PCR reaction, the magnetic beads are transported after denaturation to the opposite side of the microarray or, alternatively, out of the chamber by a magnetic field applied from the outside. In this way, the single strands react preferably or exclusively with the complementary capture molecules on the microarray.

The principal advantage of using magnetic beads in a PCR multiplex reaction is the possibility of readily producing single-stranded purified PCR products for subsequent hybridization reactions. In this way it is possible to dispense with the complicated optimization of asymmetrical PCR reactions. Compared with symmetrical PCR reactions, the yield of single-stranded PCR products is substantially increased, resulting in a higher efficiency in subsequent microarray experiments and therefore higher sensitivity of the assay.

This advantage becomes very particularly noticeable in PCR multiplexing because generating a plurality of PCR products in a single reaction is very limited here. The yields among the various PCR products often fluctuate considerably both in symmetrical and asymmetrical multiplex PCR. When using magnetic bead-coupled primers, this fluctuation is smoothed out by the fact that the amplification follows more linear rather than exponential reaction kinetics than is the case when free primers are used.

In contrast, the semi-solid phase reaction using magnetic beads addresses many of the inadequacies of a conventional symmetrical and asymmetrical PCR multiplex reaction:

Complex optimization of parameters, which is not required in multiplex reactions, in particular asymmetrical PCR multiplex reactions.

Assay speed which is accelerated in particular due to the shortened hybridization time.

The yields of single-stranded PCR products are substantially higher than in a comparable asymmetrical PCR, since all complementary DNA strands of a particular PCR product are bound to the bead surface and do not interfere with subsequent hybridization reactions.

A substantially higher degree of multiplexing, contrary to conventional homogeneous PCR reactions, is possible due to more favorable reaction kinetics.

The bead-elongated single-stranded PCR products may additionally be detected by magnetoresistive detection technologies (for example GMR or TMR sensors). This possibility enables alternative and possibly more advantageous online detection processes to be employed.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A process for detecting a plurality of target nucleic acids, comprising:
(i) providing a plurality of primer pairs including, in each case, a first and a second oligonucleotide primer suitable for amplifying said target nucleic acids, with a plurality of said first primers being coupled to a semi-solid phase support and with the second primer, in each case, being free;
(ii) providing a solution including a target nucleic acid to be detected,
(iii) contacting the primer pairs of (i) with the solution of (ii) and conducting an amplification reaction under conditions in which the target nucleic acid is amplified;
(iv) denaturing the double-stranded amplification products of (iii) to give single strands;
(v) removing the semi-solid phase support with at least one of single-stranded amplification products coupled thereto and non-extended primers coupled thereto; and
(vi) detecting the target nucleic acids.

2. The process as claimed in claim 1, wherein the semi-solid phase supports used are beads.

3. The process as claimed in claim 1, wherein the semi-solid phase supports used are magnetic beads.

4. The process as claimed in claim 1, wherein each first primer is coupled to, in each case, one semi-solid phase support.

5. The process as claimed in claim 1, wherein step (iii) is followed by a washing step to remove the primers and, where appropriate, other soluble components of the amplification reaction solution.

6. The process as claimed in claim 1, wherein the first primer is present not only coupled to the semi-solid phase support but also free in solution.

7. The process as claimed in claim 1, wherein at least one of the first and the second primer is labeled.

8. The process as claimed in claim 1, wherein the second primer, in each case, is labeled.

9. The process as claimed in claim 1, wherein detection is carried out with the aid of specific capture molecules.

10. A kit for detecting a plurality of target nucleic acids, comprising:
a plurality of primer pairs including, in each case, a first and a second oligonucleotide primer suitable for amplifying said target nucleic acid, with a plurality of said first primers being coupled to a semi-solid phase support and the second primer, in each case, being free.

11. The kit as claimed in claim 10, wherein the semi-solid phase supports are beads.

12. The kit as claimed in claim 10, wherein the semi-solid phase supports are magnetic beads.

13. The kit as claimed in claim 10, wherein each first primer is coupled to, in each case, one semi-solid phase support.

14. The kit as claimed in claim 10, wherein at least one of the first and the second primer is labeled.

15. The kit as claimed in claim 14, wherein the second primer is labeled in each case.

16. The kit as claimed in claim 10, additionally comprising specific capture molecules.

17. The kit as claimed in claim 10, further comprising reagents for carrying out an amplification reaction.

18. The kit as claimed in claim 17, wherein the semi-solid phase supports are beads.

19. The kit as claimed in claim 11, wherein the beads are magnetic beads.

20. The process as claimed in claim 2, wherein the beads used are magnetic beads.
